(19) United States
(12) Patent Application Publication
(10) Pub. No.: US 2011/0061268 A1
(43) Pub. Date: Mar. 17, 2011

(54) SOLE HAVING THE TILT SURFACE AND THE KNEE JOINT-PROTECTING SHOE COMPRISING THE SAME

(75) Inventors: Hoon Park, Seoul (KR); Hae Soo Park, Seoul (KR)

(73) Assignee: INTOOS HCN Corporation LTD., Seoul (KR)

(21) Appl. No.: 12/993,423

(22) PCT Filed: May 27, 2009
(86) PCT No.: PCT/KR09/02670

§ 371 Date: Nov. 18, 2010

(30) Foreign Application Priority Data
May 21, 2008 (KR) 2008-0047219
May 15, 2009 (KR) 2009-0042376

(51) Int. Cl. A43B 13/14; A61F 5/14

(52) U.S. Cl. 36/25 R; 36/142

(57) ABSTRACT

The present invention relates to a sole having a tilt surface, and to a knee joint-protecting shoe including same, and more particularly, to a sole having a tilt surface in which the tilt surface formed at the sole of a shoe enables the foot of a wearer to be inclined or slipped, inwardly or outwardly, in order to correct abnormality in the lower skeletal extremity, and uniformly distribute load on the knee joint of the wearer to prevent degenerative knee arthritis and alleviate pain, and to a knee joint-protecting shoe including the outsole. The present invention further comprises a sole having a tilt surface for attachment to the bottom surface of a shoe, wherein the outsole has a top surface and a bottom surface formed parallel to each other, a tilt structure including an upper member and a lower member defined by a tilt surface formed in the widthwise direction, and deformable members arranged at predetermined intervals from both front and rear ends of the shoe in a lengthwise direction to generate shear deformation in a tilted direction between the upper member and the lower member.

### TECHNICAL FIELD

The invention relates to a sole having slanted surfaces and a knee joint-protecting shoe comprising the same sole, especially it being possible for an orientation of the lower extremities skeletons of the abnormal to be corrected by the fact that a foot is tilted or slid to an inner or outer surface by the sloping surfaces being formed at the sole.

### BACKGROUND ART

A knee joint is a part of the human body to keep the balance in such a way that a human being can keep on his or her feet while bearing the weight of the body with not four feet but two feet. For this reason, the knee joint moves gradually in the moment at which we are not aware of the motion of the knee joint.

It is most ideal that a center line of the body weight must go through the center of the knee joint. However, for the most people, the center line of the body weight is not through the center of the knee joint through so that a large weight load continuously acted to only a portion of the knee joint. Accordingly, the knee joint of the body parts is one portion in which degenerative changes could be too often caused by aging. If the man has an incorrect walking habit, then he can also has an abnormal orientation of the skeletons of the legs so that the large weight could permanently affect only one part of the knee joint.

The degenerative osteoarthritis of knee joint causes symptoms such as pain in the knee when standing up, the trembling of the knees when standing, the cracking sound in the knee joint when bending or unfolding of the knee, or gap between the both knees in which more than a fist can pass in the said gap at right standing state. Further, the knee arthritis is whose existence can be doubted if there is any symptoms like pain when going down the stairs being particularly intense, or like a pain occurring for more than two or three days after walking.

The treatment of knee arthritis aims to reduce pain and increase the flexibility of the joint, but patients with such knee arthritis may not usually be cured completely. If the symptom is light, the pain and stiffness due to the ongoing can be alleviated with physical and pharmacological therapy, as well as weight reduction and the like. However, if a patient has a severe symptom of knee arthritis, the pain may be only reduced by the operation, or function of the knee joint are restored by the reconstruction. But, the operation is very expensive and annoying, so there are several cases in which the operation can not be made.

FIG. 1 is the front view showing an alignment of a right leg of the O-shaped knee, and FIG. 2 is the front view showing an alignment of a right leg of the X-shaped knee. As shown in FIGS. 1 and 2, at an alignment of a leg 1 of a person with O-shaped knee and an alignment of a leg 9 of a person with X-shaped knee, mechanical axis 50 is intended to represent the line connecting the center 3 of a femoral head with a center 5 of an ankle bone and inclined axis 70 is intended to represent the line connecting the center 3 of a femoral head with a center 7 of the knee joint.

The term “Genu Varum”, commonly called with the O-shaped knee means the bended legs with O-shaped which the inclined axis 70 is inclined relative to the mechanical axis 50 toward the outside, and then two knees are not contacted each another when standing with two legs together. On the contrary, the means of X-shaped legs standing “Genu valgum” is that the inclined axis 70 is inclined relative to the mechanical axis 50 toward the inside, and then the two legs are bent from the knee joint in the direction of the outside when standing with two legs together. The larger the value of a tilt angle β between the inclined axis 70 and the mechanical axis 50, the worse the degree of O- and X-shaped knee.

This orientation of the skeletons of the abnormal legs, such as O- and X-shaped legs, is caused from congenital and acquired factors, and the load is concentrated to only one portion of a knee joint, so the degenerative osteoarthritis of knee joint is caused or knee joint is worn and lost.

To alleviate and prevent the pain of degenerative osteoarthritis of knee joint and to correct the orientation of the skeletons of the abnormal legs, the shoe should used that is designed to tilt the foot to the inside when walking.

FIG. 3 shows the rear view of the shoes of the prior art which are worn by a person with an alignment of the skeletons of the abnormal legs or the degenerative osteoarthritis of knee joint. As shown in FIG. 3, the shoes 10 have a sole 14 and an uppers of leather 12, the sole 14 is formed so that the feet can be tilted to the inside of the shoes. If the man put on the said shoes 10 and went on the ground 500, it could be inclined the feet and legs to the inside of the shoes itself, and thus causing a rotational force to move the knee toward the inside; so that the orientation of the skeletons of the abnormal legs was improved or the degenerative osteoarthritis of knee joint was treated.

If, however, the men are putting on these shoes 10 and going on foot, there are happen excessive shear force on the ankle and knee joint because his foot are slid over to the inside of foot due to the inclination of the sole 14. So, if you have the legs excessive force so that the feet can not slide, a tremendous load acts on the inner sides of the feet and knees, which would allow the degenerative osteoarthritis of knee joint to be rather worse.

### DISCLOSURE OF THE INVENTION

### Technical problem

Starting from the said disadvantages, the invention has the objective to provide a sole with sliding surfaces and a knee joint-protecting shoe with the same sole, which the alignment of the skeletons of the abnormal legs can be corrected by inclining or sloping the foot to inward or outward with the tilt surfaces formed in the sole.

### Solution to Problem

The said objective according to the invention can be achieved by a sole having the tilt surfaces which is characterized in that, at the sole mounted to bottom of a shoe, the said sole is an inclined structure that is formed by including an upper member and a lower member, wherein the two components are split by the tilt surface being formed in the width direction.

Here, a top of the upper member and a bottom of the lower member are formed parallel to each other. The inclined structure is also designed so that the hardness of the upper member is smaller than that of the lower member. Furthermore, this inclined structure is advantageous in such a way that a deformable member is further arranged to induce shear deformation along the sloping surfaces between the upper and lower member separately. Here, the deformable member is disposed in a rest area that is excluded a certain distance from the two ends of the front and the rear side in a longitudinal direction of the shoe.

Further, the inclined structure is an arch shape that corresponds to curvature of the center of the sole of a human.

Furthermore, the inclined structure is advantageous in such a manner that the inclination is increased the tilt surfaces of the front to the rear in the longitudinal direction of the shoe.

The said inclined structure is advantageously designed so that a lowermost portion of the sloping surfaces being formed in the width direction is formed in an inside of the said sole to wear a person with O-shaped knee.

In addition, the said inclined structure is advantageously designed so that a lowermost portion of the sloping surfaces being formed in the width direction is formed in an outside of the said sole to wear a person with X-shaped knee.

Further, the inclined structure is advantageously designed so that an inner edge of the sole is formed toward an outer edge, or vice versa, the outer edge thereof is tilted toward the inner edge with a certain angle in order to form a cross section in the width direction of the sole with reverse trapezoidal shape. The angle of inclination is formed at the edge of the inner and outer edge of the sole which the lowermost part of the sloping surfaces is formed with the range of 0°<α≦60°.

In another category, the object of the invention can be achieved by a knee joint-protecting shoe, which is characterized in that the said shoe has the aforementioned sole.

The said knee joint-protecting shoes are designed so that it has an insole that is formed with the same structure as the tilted structure of the sole.

According to another category, the object of the invention can be achieved by a knee joint protective shoe, which is characterized in that, at the shoe including the insole, the said insole has the aforementioned inclined structure.

### ADVANTAGEOUS EFFECTS OF THE INVENTION

According to the invention, there is an effect which the pain of degenerative osteoarthritis of knee joint can be alleviated, and the incidence of knee arthritis can be prevented early by distributing evenly the knee distracting impacting load.

Furthermore, an effect is achieved in which an alignment of the legs of a person with O- and X-legs can be corrected to the orientation of the skeletons of the normal leg since this person can select a sole suitable for its own legs and wear the shoe being applied the said sole. Accordingly, it is ensured that the form of false legs can be corrected, so that upright walking posture and proper body structure can be established.

### BRIEF DESCRIPTION OF DRAWINGS

The above and other objects, features and advantages of the present invention will be apparent from the following detailed description of the preferred embodiments of the invention in conjunction with the accompanying drawings for understanding of technical ideas of the present invention, and hence, the present invention will not be restricted to the drawings.

FIG. 1 is the front view showing an alignment of a right leg of the O-shaped knee.

FIG. 2 is the front view showing an alignment of a right leg of the X-shaped knee.

FIG. 3 is the rear view of the shoes of the prior art which are worn by a person with an alignment of the skeletons of the abnormal legs or the degenerative osteoarthritis of knee joint.

FIG. 4 is the sectional view in the width direction of the shoe according to the first embodiment of the invention, which is worn by a person with O-shape knee.

FIG. 5 is an exploded-sectional view of the sole according to the second embodiment of the invention.

FIG. 6 is the rear view of the shoe according to the second embodiment of the invention, which is worn by a person with O-shape knee.

FIG. 7 is the rear view of the shoe according to a third embodiment of the invention.

FIG. 8 is a rear view of a state in which a person with O-shaped knee is wearing a shoe illustrated in FIG. 7 and goes on foot.

FIG. 9 is the perspective view of the sole for a person with O-shape knee according to a fourth embodiment of the invention.

FIG. 10 is the bottom view of the sole for a person with O-shape knee according to a fourth embodiment of the invention.

FIG. 11 is the left lateral view of the sole according to FIG. 10.

FIG. 12 is the right lateral view of the sole according to FIG. 10.

FIG. 13 is the sectional view along line A-A of FIG. 10.

FIG. 14 is the perspective view of the lower member according to the fifth embodiment of the invention

FIG. 15 is the bottom view of the sole for a person with O-shape knee according to a fifth embodiment of the invention.

FIG. 16 is the left lateral view of FIG. 15.

FIG. 17 is the right lateral view of FIG. 15.

FIG. 18 is the sectional view along line B-B of FIG. 15.

FIG. 19 is the sectional view along line C-C of FIG. 15.

FIG. 20 is the sectional view along line D-D of FIG. 15,

FIG. 21 is the left lateral view of the sole according to a variant form of the present invention.

FIG. 22 is the sectional view in the width direction of the shoe manufactured by the sixth embodiment of the invention.

FIG. 23 is the sectional view in the width direction of the shoe manufactured by the seventh embodiment of the invention.

FIG. 24 is the sectional view in the width direction of the shoe manufactured by the eighth embodiment of the invention.

FIG. 25 is the longitudinal section view of the knee joint-protecting shoe including insole in accordance with the ninth embodiment of the invention.

FIG. 26 is the longitudinal section view of the knee joint-protecting shoe including insole in accordance with the tenth embodiment of the invention.

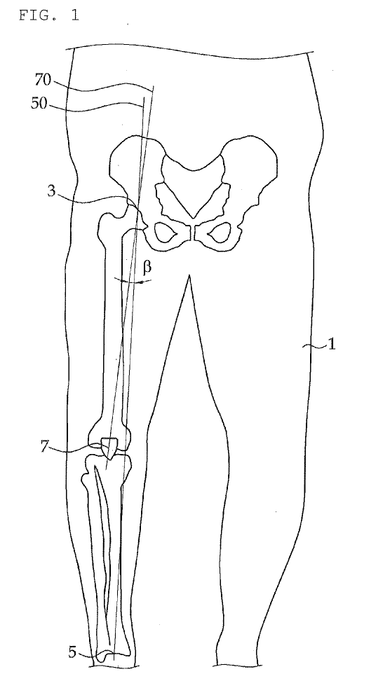

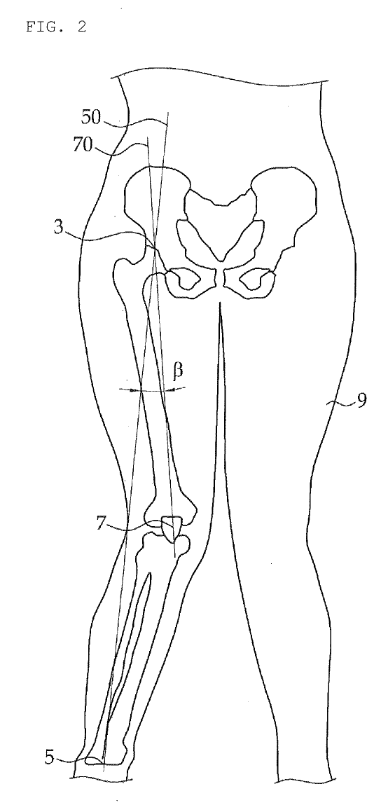

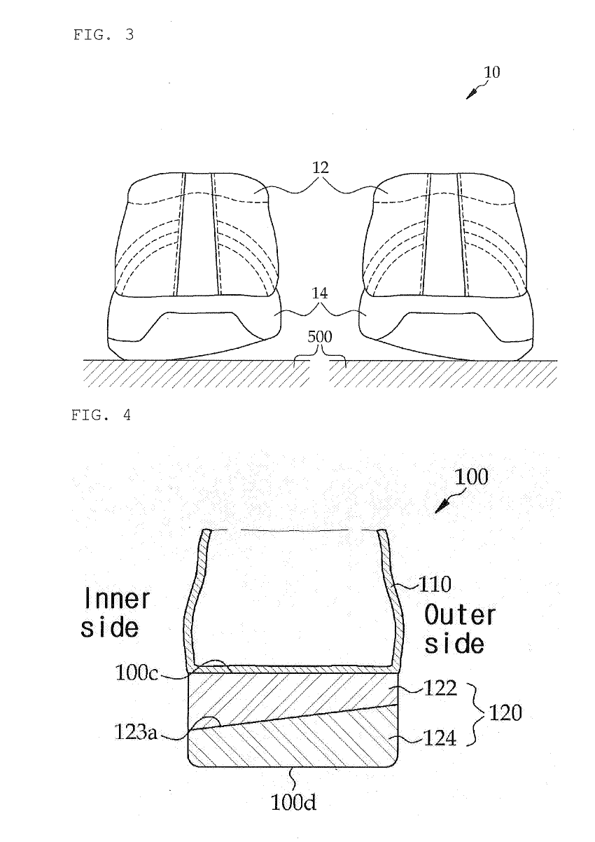

### EXPLANATION OF ESSENTIAL REFERENCE NUMERALS IN DRAWINGS

1: Orientation of the legs of a person with bow legs.
3: Center of a femoral head.
5: Center of an ankle bone.
7: Center of the knee.
9: Orientation of the legs of a person with knock knees.
10: Shoes according to the prior art.
12: Shell of the shoes according to the prior art.
14: Sole according to the prior art.
50: Mechanical axis.
70: Inclined axis
100, 200, 700, 800: Shoe
100a, 200a: Inside edge
100b, 200b: Outer edge
100c: Top.
100d: Bottom.
110, 210, 710, 810: Shell.
120, 220, 320, 420, 720, 820: Sole.
122, 322, 422: Upper member.
123, 323, 423: Deformable Member.
123a, 123b, 323a, 323b, 423a, 423b: Tilt surface.
124, 324, 424: Lower member.
250, 750, 850: Insole.
252, 752, 852: Upper member of an insole.
253, 753, 853: Deformable member of an insole.
253a, 253b, 753a, 753b, 853a, 853b: Tilt surface of an insole.
254, 754, 854: Lower member of an insole.
500: Ground.
600: Feet (a human).

### DETAILED DESCRIPTION OF THE PREFERRED EMBODIMENT

One described in the present invention, the term of “Genu Varum” means the legs being bended toward the outside with O-shaped which two knees are not contacted each other when standing with two legs together, and is generally called with O-shape knee, bow legs. On the contrary, the term of “Genu valgum” means the legs in which the two legs are bent from the knee joint in the direction of the outside when standing with two legs together, and is generally called with X-shape knee, knock-knees.

In addition, as described one in the present invention, person with bow legs covered all person who the alignment of the skeletons of legs is formed with O-shaped from congenital factors, or deformed with O-shaped from learned factors, and a pain in the inside of the knee have. Also person with the knock-knees covered all person who the alignment of the skeletons of legs is formed with X-shaped from congenital factors, or deformed with X-shaped from learned factors, and a pain in the outside of the knee have.

The following are the preferred embodiments of the invention in reference to the accompanying drawings in more detail.

### Structure of a Sole

### First Embodiment

FIG. 4 is the sectional view in the width direction of the shoe according to the first embodiment of the invention, which is worn by a person with O-shape knee, bow legs. As shown in FIG. 4, a shoe 100 includes the sole 120 and the upper leather 110, and the sole 120 of the shoe 100 is formed as an inclined structure in that it comprises an upper member 122 and a lower member 124 wherein the said two parts 122 and 124 be formed by the tilt surface 123a being split in the width direction. According to the present invention, the sole 120 of the inclined structure is made such that the tilt surface 123a is formed to be tilted toward the inside or outside of the shoe 100 according to an alignment of the skeletons of the lower limbs of the person wearing the shoe 100.

Here, the top 100c and the bottom 100d of the sole 120 are formed parallel to each other. Furthermore, the lower member 124 is made of a material with hard grade hardness than the material of the upper member 122.

As shown in FIG. 4, the sole 120 is made such that a lowermost portion of the tilt surface 123a is formed in an inner side of the sole 120, so that the shoes are worn with a person with a bow legs. Namely, If you wear the shoe 100 with the sole 120 and go on foot, the amount of deformation of inner side of the upper member 122 in which the lowermost part of the tilt surface 123a is arranged is larger than the one of outer side of the upper member 122, because the hardness of the upper member 122 is smaller than the lower member of the 124. Consequently, the foot is naturally inclined to the inside of the sole 120. Therefore, with wearing the shoe 100 and going on foot continuously, the foot is tilted to the inside of the sole 120 so that the bow leg is allowed to be corrected.

Contrary to the view of FIG. 4, the sole 120 are also advantageously prepared such that the lowermost part of the tilt surface 123a is formed in an outer side of the sole 120 in order to wear the shoes having this sole 120 with a person with X-shape knee. As being mentioned above, the amount of deformation of the outer side of the upper member 122 in which the lowermost part of the tilt surface 123a is arranged is larger than the one of inner side of the upper member 122, because the hardness of the upper member 122 is smaller than the lower member of the 124. If a person with X-shape knee wears the shoe 100 with the sole 120 and goes on foot, the foot is naturally inclined to the outside of the sole 120 so that the X-shape knee is allowed to be corrected. Furthermore, the concentrated load at to the outside of the knee is uniformly distributed to the entire knee, so that the degenerative osteoarthritis of knee joint is prevented and their symptoms can be alleviated.

### Second Embodiment

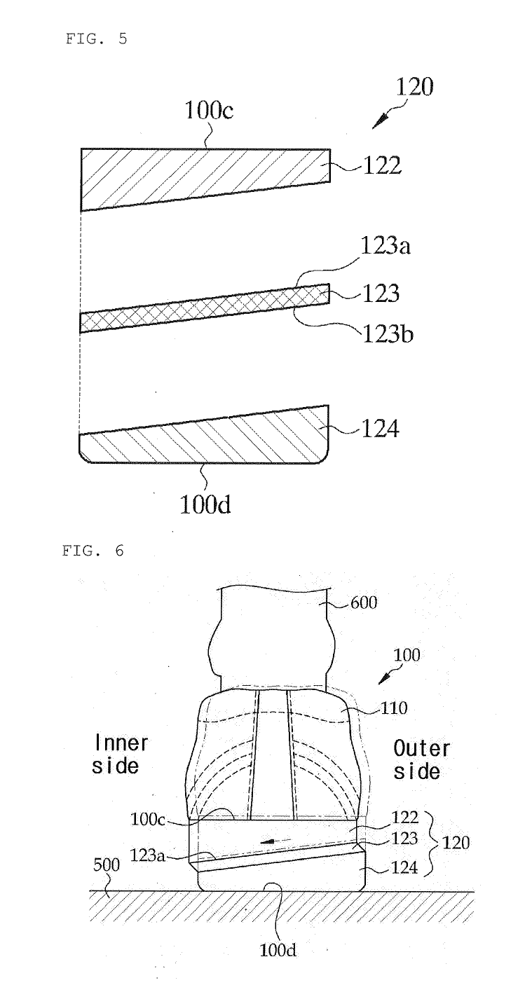

FIG. 5 is an exploded-sectional view of the sole according to the second embodiment of the invention. As shown in FIG. 5, a sole 120 is advantageously made such that a top surface 100c and a bottom surface 100d of the sole are formed parallel to each other and a deformable member 123 for inducing shear deformation along the sloping surfaces 123a and 123b is arranged between a upper member 122 and a lower member 124 to form an inclined structure. In this case, the deformable member 123 is constructed with a same inclination as in the tilt surfaces 123a and 123b, which divide the upper member 122 and lower member 124. Namely, the shear deformation of deformable member 123 is induced in the direction of the tilt surfaces 123a and 123b by force being exerted from the top surface of the sole 120, thereby acting a moment to the foot of the person wearing with the shoe 100 toward the inner or outer side while sliding the upper member 122 in the direction of the tilt surfaces 123a and 123b, so that the orientation of the skeletons of the anomalously deformed legs are corrected to normal. Here, the deformable member 123 is made from a material such as gel type synthetic resin, rubber, silicon, ethylene vinyl acetate. (EVA), polyurethane (PU), chloroprene rubber (CR) or neoprene and the like, which is deformable flexibly according to the pressure loaded on the upper member 122, so that the shear deformation of deformable member 123 is happen in the direction of the tilt surface 123.

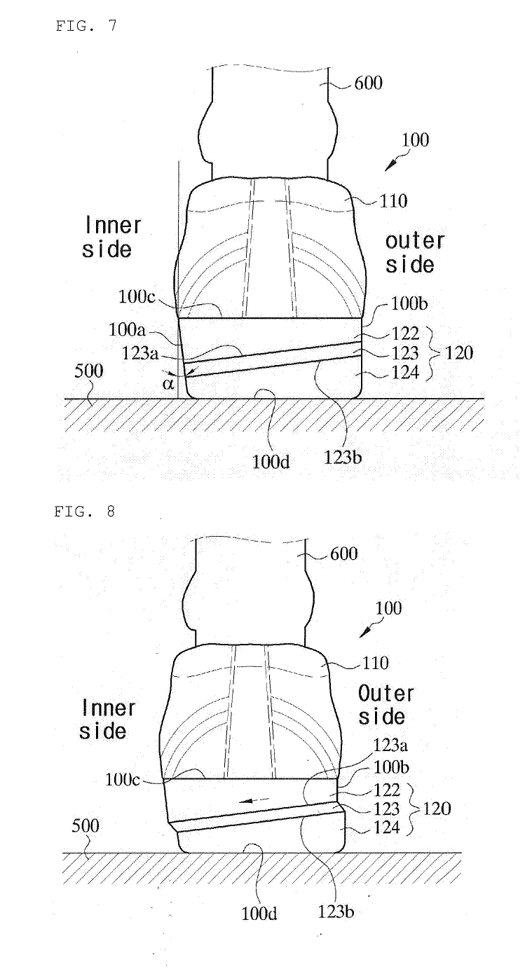

FIG. 6 is the rear view of the shoe according to the second embodiment of the invention, which is worn by a person with O-shape knee. As shown in FIG. 6, a deformable member 123 is inclined in such a manner that the upper member 122 can slide in the inner side of the sole 120, so that a person with bow legs can wear the shoe having the said sole 120. Here, the top 100c and the bottom 100d of the sole 120 are parallel to each other.

If a person with a bow leg wear the shoe 100 having the sole 120 according to the present invention and goes on foot on the ground 500, the upper member 122 may be slid toward the inside of the sole 120 by means of the applied force from above and the deformable member 123 being capable of deformation flexibly. The upper member 122 and the deformable member 123, which is illustrated with an imaginary line, may be deformed as a representation with a solid line. Furthermore, there is an eversion moment which rotates the shin to the direction of an outer side of the body of the person, while the foot 600 of the person with knock knee slides in the inside of the sole 120 by the deformation of deformable member 123.

Through this eversion moment, the distance between the right and the left knee of the person with bow legs is reduced so that these legs are to be corrected with “11”-shaped. Furthermore, the concentrated load at to the inside of the knee is uniformly distributed to the entire knee, so that the degenerative osteoarthritis of knee joint is prevented and their symptoms can be alleviated.

Contrary to the FIG. 6, the deformable member 123 is inclined such a manner that the upper member 122 can be slid into the outer side of the sole 120, so that a person with X-shape knee can wear the shoes having this sole 120. Namely, if a person with knock knees wear the shoe 100 having the sole 120 according to the present invention and goes on foot on the ground 500, the upper member 122 may be slid toward the outside of the sole 120 by means of the applied force from above and the deformable member 123 being capable of deformation flexibly. Furthermore, there is an adduction moment which rotates the shin to the direction of an inner side of the body of the person, while the foot 600 of the person with bow leg slides in the outside of the sole 120 by the deformation of deformable member 123.

Through this adduction moment, the distance between the right and the left knee of the person with X-shape knee is increased so that these legs are to be corrected with “11”-shaped. Furthermore, the concentrated load at to the outside of the knee is uniformly distributed to the entire knee, so that the degenerative osteoarthritis of knee joint is prevented and their symptoms can be alleviated.

### Third Embodiment

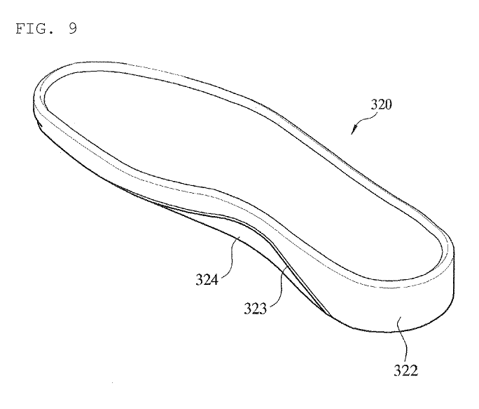

FIG. 7 is the rear view of the shoe according to a third embodiment of the invention, and FIG. 8 is a rear view of a state in which a person with O-shaped knee is wearing a shoe illustrated in FIG. 7 and goes on foot. As shown in FIGS. 7 and 8, the inclined structure is designed such that an inner edge 100a of the sole 120 is formed toward an outer edge 100b, or vice versa, the outer edge 100b thereof is tilted toward the inner edge 100a with a certain angle α in order to form a cross section in the width direction of the sole 120 with reverse trapezoidal shape. The angle of inclination α is formed at the edge of the sole 120 which the lowermost part of the sloping surfaces 123a and 123b is formed with the range of 0°<α≦60°.

As shown in FIG. 7, the sole 120 which consists of the upper member 122, the deformable member 123 and the lower member 124 makes it possible for person with bow legs to wear the shoes having this sole 120 which the lowest part of the sloping surfaces 123a and 123b is formed at the inner side of the sole 120 by tilting the inner edge 100a with the tilt angle α relative to the outer edge 100b when placing the shoe 100 on the ground 500. A section in the width direction of the sole 120 has a reverse trapezoidal shape. The angle of inclination α can be regulated according to the bending degree of O-shape knee. If the angle of inclination α exceeds 60°, the change in direction on the inside of the sole 120 may be even stronger, so it is advantageous that the angle of inclination does not exceed 60°. According to the present invention, the sole 120 is designed such that the angle of inclination α is 15°. As shown in FIG. 8, the inner edge 100a of the sole 120 is formed with the angle of inclination α relative to the outer edge 100b so that the center of gravity for the upper member 122 and the lower member 124 does not match. Therefore, the upper member 122 is slid into inside of the sole 120 toward the direction of the tilt surfaces 123a and 123b of the deformable member 123 to occur deformation. Namely, the upper member 122 slides in the direction of the tilt surfaces 123a and 123b of the deformable member 123 and hence the foot 600 of the person with bow legs is slid into the inside of the sole 120 so that the orientation of the skeletons of the abnormal legs can be corrected.

Contrary to the FIG. 7, the sole 120 which consists of the upper member 122, the deformable member 123 and the lower member 124 makes it possible for person with knock knees to wear the shoes having this sole 120 which the lowest part of the sloping surfaces 123a and 123b is formed at the outer side of the sole 120 by tilting the outer edge 100b with the predetermined tilt angle α relative to the inner edge 100a when placing the shoe 100 on the ground 500. Here, the angle of inclination α is to the range of 0°<α≦60° and a section in the width direction of the sole 120 has a reverse trapezoidal shape. If the angle of inclination α exceeds 60°, the change in direction on the outside of the sole 120 may be even stronger, so it is advantageous that the angle of inclination does not exceed 60°.

Contrary to the FIG. 7, the outer edge 100b of the sole 120 is formed with the angle of inclination α relative to the inner edge 100a so that the center of gravity for the upper member 122 and the lower member 124 does not match. Therefore, the upper member 122 is slid into outside of the sole 120 toward the direction of the tilt surfaces 123a and 123b of the deformable member 123 to occur deformation. Namely, the upper member 122 slides in the direction of the tilt surfaces of the deformable member 123 and hence the foot 600 of the person with knock knees is slid into the outside of the sole 120 so that the orientation of the skeletons of the abnormal legs can be corrected.

The sole 120 designed according to the present invention can correct the orientation of the skeletons of the abnormal legs normally, and evenly distribute the concentrated load applied to only one portion of the knee joint so that the degenerative osteoarthritis of knee joint is prevented and the pain can be alleviated.

If the angle of inclination α as the third embodiment according to the invention is applied to the first embodiment, a sole 120 with an inverted trapezoidal section also can be designed.

### Fourth Embodiment

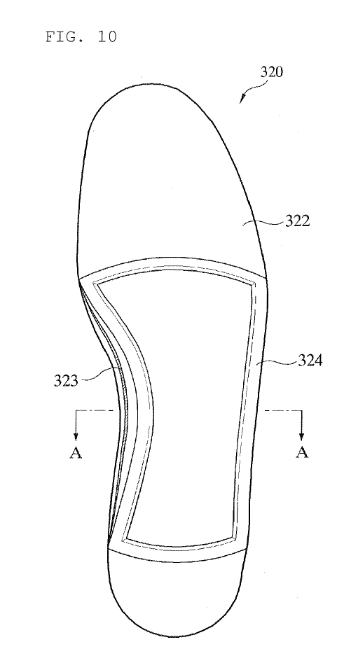

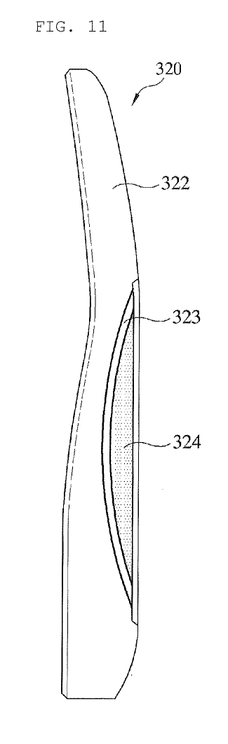

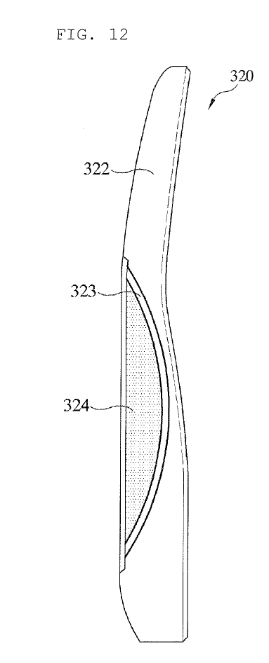

FIG. 9 is the perspective view of the sole for a person with O-shape knee according to a fourth embodiment of the invention, FIG. 10 is the bottom view of the sole for a person with O-shape knee according to a fourth embodiment of the invention, FIG. 11 is the left lateral view of the sole according to FIG. 10, and FIG. 12 is the right lateral view of the sole according to FIG. 10. As shown in FIGS. 9 to 12, the sole according to the fourth embodiment of the present invention consists of an upper member 322, a lower member 324 and a deformable member 323 arranged between the upper member 322 and lower member 324.

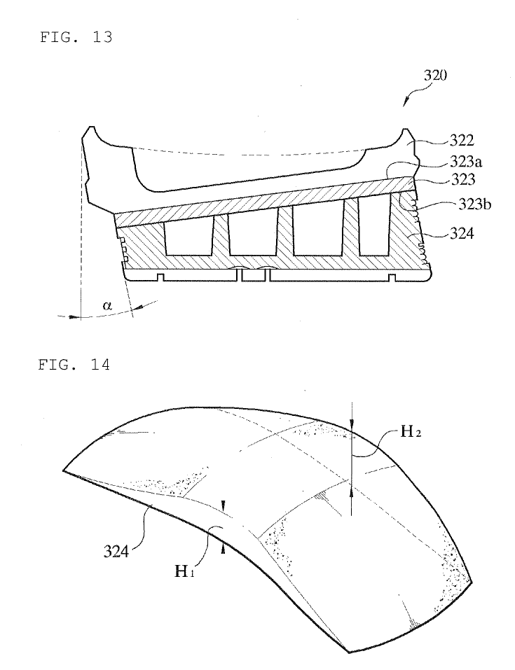

The sole 320 according to the fourth embodiment of the present invention, as shown in FIGS. 9 to 12, designed such that on one side of a bottom of the sole 320, an arc-shaped dome is arranged with a shape that corresponds to the arc-part being at the middle of the man's sole. It is advantageous that this arc-shaped dome is located at one side of the center of the shoe, preferably at one place where corresponds to the arc-part being at the man's sole. FIG. 13 is the sectional view along line A-A of FIG. 10, and FIG. 14 is the perspective view of the lower member according to the fourth embodiment of the invention. The lower member 324 of the fourth embodiment of the present invention as shown in FIGS. 13 and 14 is designed such that its base is generally parallel to the ground and its top has arcuate shape along with a longitudinal direction of a sole 320. Furthermore, the tilt surfaces 323a and 323b is formed to be higher one side of the inside or outside than the other side in the width direction, as shown in FIG. 13.

Such tilt surfaces 323a and 323b is formed such that one side of the inside or outside is higher than the other side according to an orientation of the skeleton of the legs of a person wearing the shoes. In one embodiment, the ratio of the height H1 of the inside to the height H2 of the outside in the width direction of the sole 320 is 1:1.5 to 1:2. Namely, if the height H1 of the inside is 1 cm, the height H2 of the outer side is 1.5 cm to 2 cm. Here, the direction and height of tilt surfaces 323a and 323b may be varied in accordance with the orientation of the skeletons of legs and the pain of the knee of the person wearing the shoes. In addition, the lower member 324 is made of a material with a greater degree of performing a hard hardness than the material of the upper member 322.

As shown in FIGS. 13 and 14, the sole 320 is made such that a lowermost portion of the tilt surface 323b of the lower member 324 is formed in an inner side of the sole 320, so that the shoes are worn with a person with a bow legs. Namely, If you wear the shoe with the sole 320 and go on foot, the amount of deformation of inner side of the sole 320 in which the lowermost part of the tilt surface 323a and 323b is arranged is larger than the one of outer side, because the overall hardness of the upper member 322 is smaller than the lower member of the 324. Consequently, the foot is naturally inclined to the inside of the sole 320. Therefore, with wearing the shoe having the sole 320 according to the present invention and going on foot continuously, the knee and the foot are tilted to the inside of the body so that the bow leg is allowed to be corrected.

On the other hand, it is envisaged that the lowest part of the tilt surface 232b of the lower member 324 may be formed at the outer side of the sole 320 in order to wear it with a person with knock knee, thereby being tilted the feet of the person wearing the shoes toward the outside of the sole 320 according to the same principle with the said sole 320 for the person with bow legs.

With the sole 320 having the above construction, the O- and X-shape knee can be corrected, and the load focused on the inside or the outside of the knee is distributed over the entire knee, so that degenerative osteoarthritis of knee joint is prevented and their symptoms can be alleviated.

The deformable member 323 according to the fourth embodiment of the present invention causes a shear deformation forward inclined direction of the tilt surfaces 323a and 323b. In this case, the deformable member 323 is constructed with a same inclination as in the tilt surfaces 323a and 323b, which divide the upper member 322 and lower member 324. Namely, the deformable member 323 is slid along with the tilt surfaces 323a and 323b by force being exerted from the top surface of the sole 320, thereby acting a moment to the foot of the person wearing with the shoe toward the inner or outer side while sliding the upper member 322 in the direction of the tilt surfaces 323a and 323b, so that the orientation of the skeletons of the anomalously deformed legs are smoothly corrected to normal. The materials of such deformable member 323 may be selected from gel type synthetic resin, rubber, silicon, ethylene vinyl acetate, polyurethane or chloroprene rubber, and the like.

As shown in FIG. 13, for more smoother correct, the sole 320 having the abovementioned construction is designed such that an inner edge of the sole 320 is formed toward an outer edge, or vice versa, the outer edge thereof is tilted toward the inner edge with a certain angle α in order to form a cross section in the width direction of the sole 320 with reverse trapezoidal shape. The angle of inclination α may preferably be in the range between 0° and 60°. This angle of inclination α can be varied according to the condition of the knee and the pain of a person wearing the shoes. However, it is more advantageous that the angle of inclination α is 10° to 20°. If the angle of inclination exceeds 60°, the change of the sole 320 are even stronger, so it is advantageous that the angle of inclination α does not exceed 60°.

To wear the shoe with a person with O-shape knee, as shown in FIG. 13, the bottom of inner edge is offset relative to the outer edge with the angle of inclination α so that the center of gravity of the upper member 322 the lower member 324 is not matched, thereby the sole 320 more easily sliding to the inside of the body by the shear deformation of deformable member 323, so the O-shape knee can be corrected.

Further, the bottom of outer edge is offset relative to the inner edge by the angle of inclination α to put on it with a person having X-shape knee, contrary to the FIG. 13. Being formed the slope in this way, the center of the body slides to the outside of the body when walking, so the knock-knees can be corrected.

### Fifth Embodiment

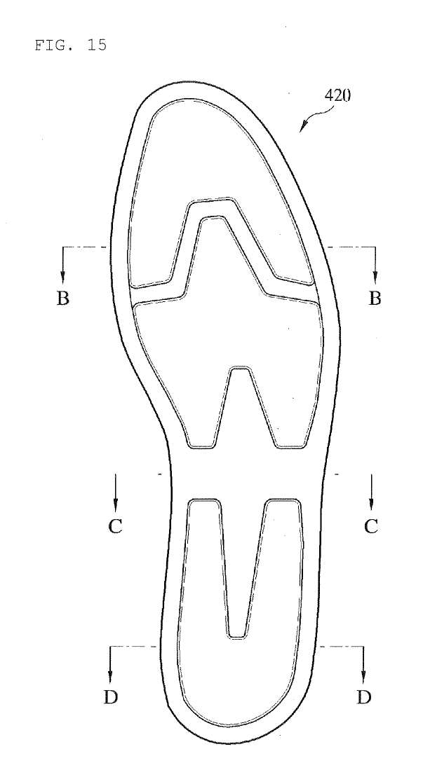

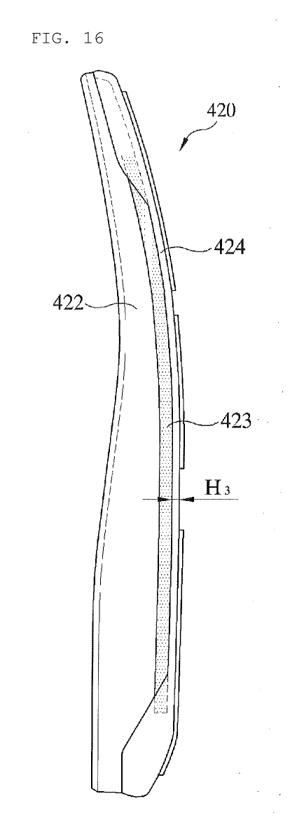

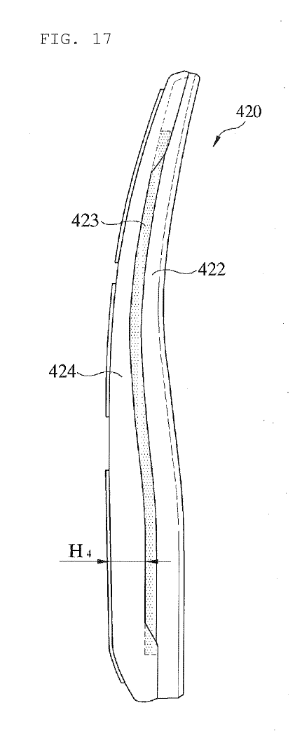

FIG. 15 is the bottom view of the sole for a person with O-shape knee according to a fifth embodiment of the invention, FIG. 16 is the left lateral view of FIG. 15, and FIG. 17 is the right lateral view of FIG. 15. As shown in FIGS. 15 to 17, the sole 420 according to the fifth embodiment of the present invention consists of an upper member 422, a lower member 424 and a deformable member 423 arranged between the upper member 422 and lower member 424.

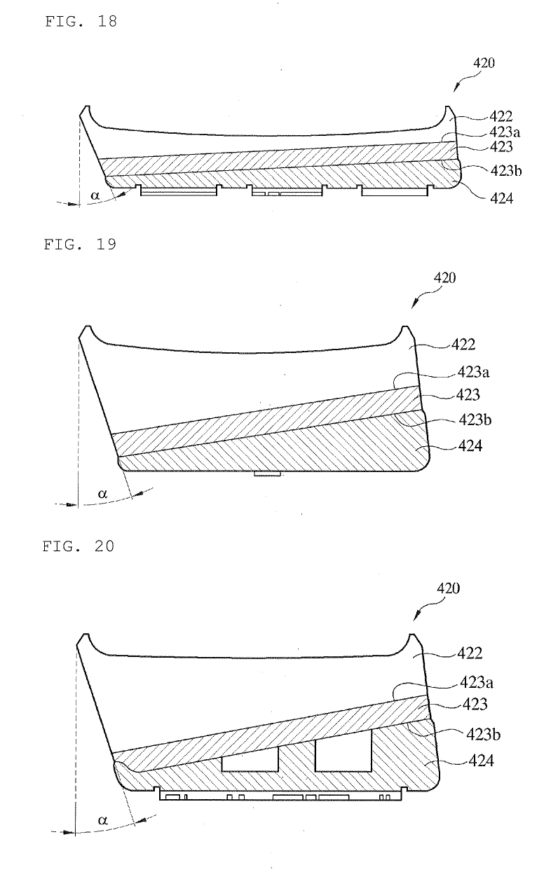

FIG. 18 is the sectional view along line B-B of FIG. 15, FIG. 19 is the sectional view along line C-C of FIG. 15, and FIG. 20 is the sectional view along line D-D of FIG. 15. As shown in FIGS. 18 to 20, the sole 420 according to the fifth embodiment of the invention is formed with an inclined structure, which includes an upper member 422 and a lower member 424 formed by a deformable member 423 cleaved with width direction. According to the present invention, the sole 420 of the inclined structure is made such that the deformable member 423 is formed to slide to an inner or an outer side of the shoes depending on the orientation of the skeletons of the legs of a person wearing the shoes.

It has a top and a bottom of the sole 420, namely a top of the upper member 422 and a bottom of the lower member 424 are formed parallel to each other. Further, the lower member 424 is made of a material with a greater hardness than the material of the upper member 422.

According to FIGS. 15 to 20, the sole 420 is made such that a lowermost portion of the deformable member 423 is formed in an inner side of the sole 420 to enable a person with bow legs wearing the shoes produced from it. Therefore, if you wear the shoes having this sole 420 and goes on foot, the foot is naturally slid into an inside of the sole 420 because the amount of deformation of inner side of the upper member 422, in which the lowest part of the deformable member 423 is arranged is larger than that of an outside of the upper member 422 due to the smaller hardness the upper member 422. In one embodiment, the ratio of the height H3 of the inside to the height H4 of the outside in the width direction of the sole 420 is 1:1.5 to 1:2. Namely, if the height H3 of the inside is 1 cm, the height H4 of the outer side is 1.5 cm to 2 cm. If you keep on this way with the shoes and goes on foot, the foot is tilted to the inside of the sole 420, so that the bow-leg to be corrected. To increase the effect of correcting the legs, it is also advantageous that the inclination of the tilt surfaces 423a and 423b can be increased from the front to the rear in the longitudinal direction of the shoe. But it is obvious that the inclination of the front can still change bigger according to a level of the bent legs and a knee pain.

On the other hand, it is envisaged that the lowest part of the deformable member 423 may be formed at the outer side of the sole 420 in order to wear it with a person with knock knee, thereby being tilted the feet of the person wearing the shoes toward the outside of the sole 420 according to the same principle with the said sole 420 for the person with bow legs.

With the sole 420 having the above construction, the O- and X-shape knee can be corrected, and the load focused on the inside or the outside of the knee is distributed over the entire knee, so that degenerative osteoarthritis of knee joint is prevented and their symptoms can be alleviated.

The deformable member 423 according to the fifth embodiment of the present invention is arranged between the upper member 422 and lower member 424 to cause a shear deformation forward inclined direction of the deformable member 423. Here, the deformable member 423 disposed in a rest area that is ruled by each of the two ends of the front and the rear side in a longitudinal direction of the shoe by a predetermined distance. The reason, in which the deformable member is positioned 423 in the remaining area, is to seek to ensure that a strain of the ankle and knee, caused by the fact that the feet are totally inclined to the inside or the outside, could be reduced. In this case, the deformable member 423 is constructed with a same slant as in the tilt surfaces 423a and 423b, which split the upper member 422 and the lower member 424 of each other, wherein the deformable member 423 is disposed in a central region of the shoe which is excluded by 5% to 15% from each side of the ends of the front and the rear side in the longitudinal direction of the shoe. Namely, the deformable member is slid along with the inclined direction by force being exerted from the top surface of the sole 420, thereby acting a moment to the foot of the person wearing with the shoe toward the inner or outer side while sliding along with the direction of the tilt surfaces, so that the orientation of the skeletons of the anomalously deformed legs are smoothly corrected to normal. Also, a strain of the ankle and knee can be reduced since the sliding part with the inclined direction is taken at the ends of the front and the rear side in the longitudinal direction of the shoe. The materials of such deformable member 423 may be selected from gel type synthetic resin, rubber, silicon, ethylene vinyl acetate, polyurethane, chloroprene rubber, and the like.

As shown in FIGS. 18 to 20, for more smoother correct, the sole 420 having the abovementioned construction is designed such that an bottom of inner edge of the sole 420 is offset toward an outer edge, or vice versa, the bottom of outer edge thereof is offset toward the inner edge with a certain angle in order to form a cross section in the width direction of the sole 420 with reverse trapezoidal shape.

The angle of inclination α may preferably be in the range between 0° and 60°. This angle of inclination α can be varied according to the condition of the knee and the pain of a person wearing the shoes. However, it is more advantageous that the angle of inclination α is 10° to 20°. If the angle of inclination exceeds 60°, the change of the sole 420 are even stronger, so it is advantageous that the angle of inclination α does not exceed 60°. To wear the shoe with a person with O-shape knee, as shown in FIGS. 18 to 20, the bottom of inner edge is offset relative to the outer edge with the angle of inclination α so that the center of gravity of the upper member 422 and the lower member 424 is not matched, thereby the sole 420 more easily sliding to the inside of the body by the shear deformation of deformable member 423, so the O-shape knee can be corrected.

Further, the bottom of outer edge is offset relative to the inner edge by the angle of inclination α to put on it with a person having X-shape knee, contrary to the FIGS. 18 to 20. Being formed the slope in this way, the center of the body slides to the outside of the body when walking, so the knock-knees can be corrected.

### Variant Embodiment

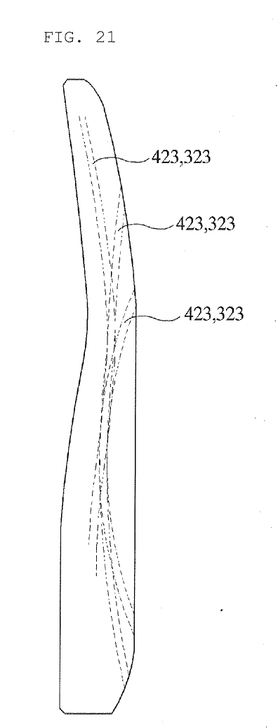

FIG. 21 is the left lateral view of the sole according to a variant form of the present invention. As shown in FIG. 21, a deformable member 323, 423 of a sole 320, 420 according to the present invention can be changed depending on a curvature form a sole of a person and a whole shape of a shoe, as well as a deformation and a health condition of the knee of the person.

### <Structure of Knee Joint-Protecting Shoes>

The invention provides a generic, preferably knee joint-protecting shoe which is advantageously constructed by connecting a sole 120 to 420 according to the first to the fifth embodiment to this sole with means of all types of adhesives or sewing, and the like. With wearing shoes made from the said method and going on foot, the O- or X-shape knee may be corrected to “II-shape” leg without any operation.

The shoe for protecting a knee joint according to the present invention may further include a insole 250, 750 or 850. In addition, the said insole 250, 750 or 850 is designed as follows:

### Sixth Embodiment

FIG. 22 is the sectional view in the width direction of the shoe manufactured by the sixth embodiment of the invention. As shown in FIG. 22, the shoe 200, in which a sole 220 is attached to an underside of an leather 210 and the insole 250 is placed in the leather 210, is preferably constructed such that the insole 250 contain an upper member 252 and a lower member 254, these two parts are formed by a fissured tilt surface 253a in the width direction. Namely, the insole 250 is made as an equal inclined structure as in the first embodiment described at the above. FIG. 22 shows a sectional view of the shoe for a person with O-shape, but the person with O- or X-shape knee can choose a suitable insole depending on a position of a bottom portion of the tilt surface 253a, that is depending on whether this bottom part is formed in an inside or an outside of the shoe 200. Furthermore, the hardness of the upper member 252 and the lower member 254 is different from each other as in the first embodiment.

In this case, as the sole 220, a generic sole or any sole 120, 320, 420, 720 or 820 according to the first through the fifth embodiment of the present invention may be used.

### Seventh Embodiment

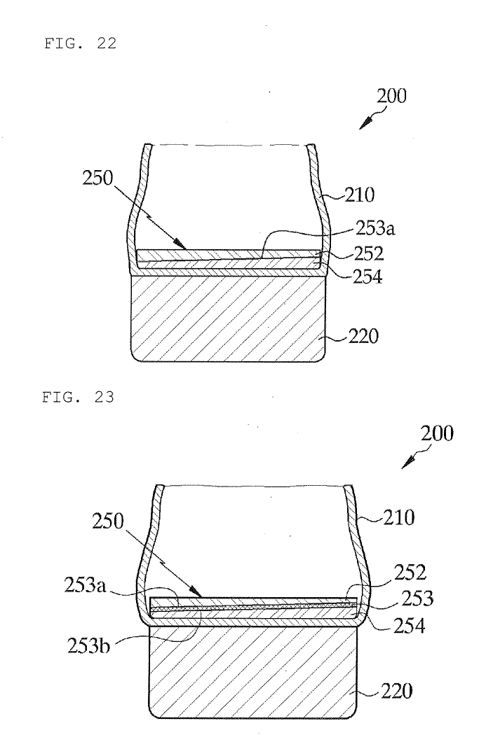

FIG. 23 is the sectional view in the width direction of the shoe manufactured by the seventh embodiment of the invention. As shown in FIG. 23, an insole 250 is advantageously designed such that it additionally includes a deformable member 253, which induces a shear deformation along the sloping surfaces 253a and 253b between the upper member 252 and the lower member 254. Namely, the insole 250 is made as an equal inclined structure as in the second embodiment described at the above. FIG. 23 shows a sectional view of the shoe for a person with O-shape, but the person with O- or X-shape knee can choose a suitable insole depending on a position of a bottom portion of the tilt surface 253a and 253b, that is depending on whether this bottom part is formed in an inside or an outside of the shoe 200.

In this case, as the sole 220, a generic sole or any sole 120, 320, 420, 720 or 820 according to the first through the fifth embodiment of the present invention may be used.

### Eighth Embodiment

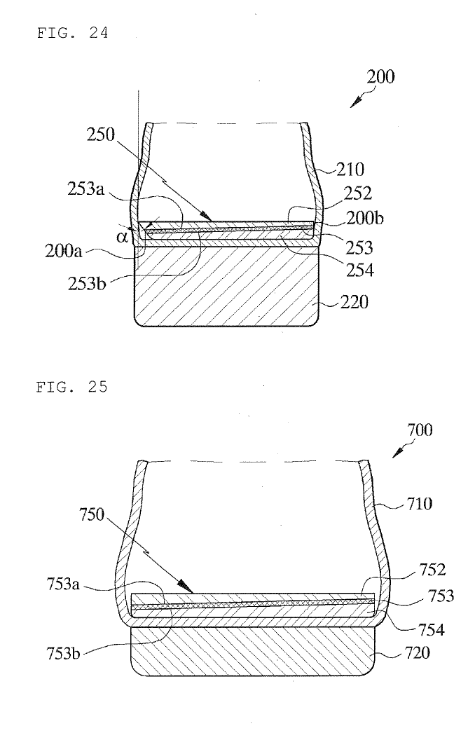

FIG. 24 is the sectional view in the width direction of the shoe manufactured by the eighth embodiment of the invention. As shown in FIG. 24, the inclined structure is designed such that a bottom of inner edge 200a of the insole 250 is offset toward an outer edge 200b, or vice versa, the bottom of outer edge 200b thereof is offset toward the inner edge 200a with a certain angle α in order to form a cross section in the width direction of the insole 250 with reverse trapezoidal shape. The angle of inclination α is formed at the edge which the lowermost part of the sloping surfaces 253a and 253b is formed with the range of 0°<α≦60°.

Namely, the insole 250 is made as an equal inclined structure including an angle of inclination α as in the third embodiment described at the above. FIG. 24 shows a sectional view of the shoe for a person with O-shape, but the person with O- or X-shape knee can choose a suitable insole depending on a position of a bottom portion of the tilt surface 253a and 253b, that is depending on whether this bottom part is formed in an inside or an outside of the shoe 200.

In this case, as the sole 220, a generic sole or any sole 120, 320, 420, 720 or 820 according to the first through the fifth embodiment of the present invention may be used. It is also possible to form the insole 250 having an inverted trapezoidal section by applying the same angle of inclination α with the eighth embodiment of the invention to the sixth embodiment.

### Ninth Embodiment

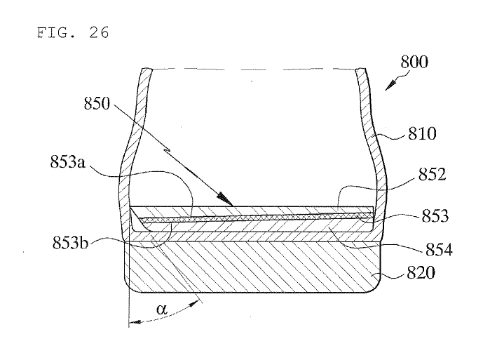

FIG. 25 is the longitudinal section view of the knee joint-protecting shoe including insole in accordance with the ninth embodiment of the invention. The insole 750 according to the ninth embodiment of the invention includes the deformable member 753 to induce a shear deformation along with the upper member 752, the lower member 754, and the sloping surfaces 753a and 753b between the upper member 752 and the lower member 754 which is formed by the tilt surfaces splitted in the width direction. Namely, the insole 750 is made as an equal inclined structure with the inclined structure of the insole 320, 420 according to the fourth or the fifth embodiment described at the above. This inclined structure of the insole 750 may change with considering the light of the knee condition and pain level of a person wearing the shoes as diverse as that of the insole 320 or 420 according to the fourth or the fifth embodiment of the invention.

With this insole 750 being formed to have the same inclined structure with the sole, it can be obtained the effect which the legs are correct, and prevent the degenerative knee arthritis and relieve their pain.

In this case, as the sole 720 of the shoe 700 according to the ninth embodiment, a generic sole or any sole 320 or 420 according to the first through or fifth embodiment of the present invention may be used.

Here, the non-described reference numeral 710 is a shell of a shoe.

### Tenth Embodiment

FIG. 26 is the longitudinal section view of the knee joint-protecting shoe including insole in accordance with the tenth embodiment of the invention. The insole 850 according to the tenth embodiment of the invention, as seen in FIG. 26, includes the deformable member 853 to induce a shear deformation along with the upper member 852, the lower member 854, and the sloping surfaces 853a and 853b between the upper member 852 and the lower member 854 which is formed by the tilt surfaces splitted in the width direction, with the same inclined structure as that of the insole 850 according to the ninth embodiment. Also, the insole 850 is designed such that a bottom of inner edge of the sole 850 is offset toward an outer edge, or vice versa, the bottom of outer edge thereof is offset toward the inner edge with a certain angle α in order to form a cross section in the width direction of the sole 850 with reverse trapezoidal shape. The angle of inclination α may preferably be in the range between 0° and 60° such as the sole 320 according to the fourth embodiment or the sole 420 according to the fifth embodiment. This angle of inclination α can be varied according to the condition of the knee and the pain of a person wearing the shoes 800. However, it is more advantageous that the angle of inclination α is 10° to 20°. If the angle of inclination exceeds 60°, the change of the sole 850 are even stronger, so it is advantageous that the angle of inclination α does not exceed 60°.

In this case, as the sole 820 of the shoe 800 according to the tenth embodiment, a generic sole or any sole 320 or 420 according to the first through or fifth embodiment of the present invention may be used.

Here, the non-described reference numeral 810 is a shell of a shoe.

As mentioned above, it is quite understandable for a person of ordinary skill of the present invention that the invention can be carried out without changing the essential features and technical ideas in a different concrete form. Therefore, it must be understood that the above-mentioned embodiments are merely way of example, and thus the present invention is not limited thereto. It must also be interpreted that the scope of the present invention is not limited by the foregoing description but only by the below described patent claims.

What is claimed is:

1. Sole 120, 220, 320, 420, 720, 820 with tilt surfaces, which is attached to a bottom of a shoe 100, 200, 700, 800, wherein the sole 120, 220, 320, 420, 720, 820 has an inclined structure having an upper member 122, 322, 422 and a lower member 124, 324, 424, wherein the two parts are split by the tilt surfaces 123a, 323a, 323b, 423a, 423b being formed with the width direction.

2. Sole with the tilt surfaces according to claim 1, wherein the sole is designed such that a top surface 100c of the upper member 122, 322, 422 and a bottom surface 100d of the lower member 124, 324, 424 are formed parallel to each other.

3. Sole with the tilt surfaces according to claim 1, wherein the inclined structure is designed such that the hardness of the upper member 122, 322, 422 is less than that of the lower member 124, 324, 424.

4. Sole with the tilt surfaces according to claim 1, wherein the inclined structure further includes a deformable member 123, 323, 423, wherein the deformable member induces a shear deformation along with the tilt surfaces 123a, 323a, 323b, 423a, 423b between the upper member 122, 322, 422 and the lower member 124, 324, 424.

5. Sole with the tilt surfaces according to claim 4, wherein the deformable member 123, 323, 423 is located in the rest area that is ruled by each of the two ends of the front and the rear side in a longitudinal direction of the shoe by a certain distance.

6. Sole with the tilt surfaces according to claim 1, wherein the inclined structure is an arched form, the form being given to correspond to the middle of the plantar arch of a human.

7. Sole with the tilt surfaces according to claim 1, wherein the inclined structure is designed such that the angle of inclination of the tilt surfaces 123a, 323a, 323b, 423a, 423b is increased by going from the front to the rear in the longitudinal direction of the shoe.

8. Sole with the tilt surfaces according to claim 1 or 4, characterized in that the inclined structure is constructed such that a lowermost part of the tilt surfaces 123a, 323a, 323b, 423a, 423b being formed in the width direction is arranged in an inner side of the sole 120, 220, 320, 420, 720, 820, for a person with bow legs.

9. Sole with the tilt surfaces according to claim 1 or 4, characterized in that the inclined structure is constructed such that a lowermost part of the tilt surfaces 123a, 323a, 323b, 423a, 423b being formed in the width direction is arranged in an outer side of the sole 120, 220, 320, 420, 720, 820, for a person with knock knees.

10. Sole with the tilt surfaces according to claim 1 or 4, characterized in that the inclined structure is constructed such that the bottom of inner edge 100a of the sole 120, 320, 420 is offset toward an outer edge 100b thereof, or vice versa, the bottom of outer edge 100b thereof is offset toward the inner edge 100a with a certain angle α in order to form a cross section in the width direction of the sole 120, 320, 420 with reverse trapezoidal shape, the angle of inclination α is formed with the range of 0°<α≦60°, and is formed in one edge of the inner edge 100a and outer edge 100b of the sole 120, 320, 420, in which the lowermost part of the tilt surfaces 123a, 323a, 323b, 423a, 423b is arranged.

11. Knee joint-protecting shoe characterized in that the shoe includes the sole 120, 320, 420 according to the claim 10.

12. Knee joint-protecting shoe according to claim 11, characterized in that the shoe is designed such that the shoe includes the insole having the same structure with the inclined structure of the sole 120, 320, 420.

13. Shoe 200, 700, 800 with an insole 250, 750, 850, wherein the insole 250, 750, 850 has the inclined structure according to the claim 10.
